(19) United States
(12) Patent Application Publication
(10) Pub. No.: US 2012/0225926 A1
(43) Pub. Date: Sep. 6, 2012

(54) AMPHIPHILIC MACROMOLECULES FOR NUCLEIC ACID DELIVERY

(76) Inventors: Kathryn E. Uhrich, New Brunswick, NJ (US); Sarah M. Sparks, New Brunswick, NJ (US); Li Gu, New Brunswick, NJ (US); Alex Harmon, New Brunswick, NJ (US); Charles M. Roth, New Brunswick, NJ (US); Carolyn Waite, New Brunswick, NJ (US)

(21) Appl. No.: 13/402,793

(22) Filed: Feb. 22, 2012

Related U.S. Application Data
(60) Provisional application No. 61/445,401, filed on Feb. 22, 2011.

(51) Int. Cl. A61K 31/711; C12N 5/02; A61K 31/713; A61K 31/7105; C08G 69/40; C08G 69/48

(52) U.S. Cl. 514/44 A; 528/322; 525/435; 514/44 R; 435/375

(57) ABSTRACT

The invention provides amphiphilic macromolecules that are useful for delivering nucleic acids to cells and that are useful as delivery agents for gene therapy.

### CROSS-REFERENCE TO RELATED APPLICATION(S)

This patent application claims the benefit of priority of U.S. application Ser. No. 61/445,401, filed Feb. 22, 2011, which application is herein incorporated by reference.

### BACKGROUND OF THE INVENTION

The use of novel amphiphilic macromolecules (AMs) for drug delivery has been an area of recent interest. Nanoscale amphiphilic macromolecules (AMs) are biocompatible amphiphilic polymers composed of an alkylated sugar backbone covalently linked to poly(ethylene glycol) (PEG). In aqueous solution, AMs self-assemble to form 10-20 nm micelles with critical micelle concentrations as low as 100 nM, making them more stable than other common micelles. In addition, the basic structure of AMs has multiple points of modification such that the polymer can be modified and evaluated for virtually any application.

These polymers were first designed and synthesized by L. Tian, L. Yam, N. Zhou, H. Tat, and K. Uhrich, Macromolecules 2004, 37, 538. Since their initial synthesis, AMs have been evaluated for the delivery of cancer therapeutics and for the inhibition of highly oxidized low-density lipoprotein in macrophage cells.

Currently, the most efficacious systems to deliver nucleic acids are viruses. However, as they are viruses, they may be unsafe for use as they will provoke an immune reponse. Thus, novel, versatile, and effective synthetic systems to deliver nucleic acids are needed. Currently, there is a lack of effective systems that are biocompatible and versatile.

### SUMMARY OF THE INVENTION

Applicant has now functionalized AMs for nucleic acid delivery. The AMs of the invention are biocompatible and are useful for delivering a variety of nucleic acids efficiently.

In one embodiment the invention provides a compound of formula I:

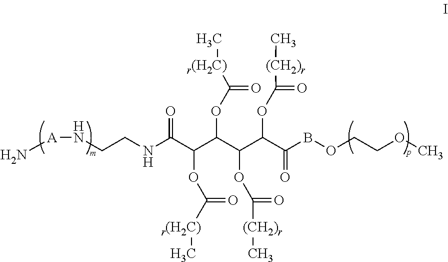

wherein:

B is absent or the group —NH(-A-NH—)ₙ—(CH₂)₂—NH—C(═O)—CH₂—;

each A is independently an ethylene group that is optionally substituted with one or more (C₁-C₃)alkyl groups;

m is an integer from 0 to 500;

n is an integer from 0 to 500;

p is an integer from 1 to 500; and each r is independently an integer from 0 to 20; or a salt thereof.

In another embodiment the invention provides a composition comprising a compound of formula I or a salt thereof and a nucleic acid (e.g. DNA, RNA or siRNA).

In another embodiment, the invention provides a pharmaceutical composition comprising a compound of formula I or a pharmaceutically acceptable salt thereof and a nucleic acid (e.g. DNA, RNA or siRNA) and a pharmaceutically acceptable carrier.

In another embodiment, the invention provides a composition comprising a compound of formula I or a salt thereof and a lipid and a nucleic acid (e.g. DNA, RNA or siRNA).

In another embodiment, the invention provides a pharmaceutical composition comprising a compound of formula I or a pharmaceutically acceptable salt thereof and a lipid and a nucleic acid (e.g. DNA, RNA or siRNA) and a pharmaceutically acceptable carrier.

In another embodiment, the invention provides a method for delivering a nucleic acid (e.g. DNA, RNA or siRNA) into a cell comprising contacting the cell with a composition comprising a compound of formula I or a salt thereof and the nucleic acid under conditions such that the nucleic acid is delivered into the cell.

In another embodiment, the invention provides a method for delivering a nucleic acid (e.g. DNA, RNA or siRNA) into a cell comprising contacting the cell with a composition comprising a compound of formula I or a salt thereof and a lipid and the nucleic acid under conditions such that the nucleic acid is delivered into the cell.

The invention also provides processes and intermediates disclosed herein that are useful for preparing compounds of formula (I) or salts thereof.

### BRIEF DESCRIPTION OF THE DRAWINGS

FIG. 1 shows the U87 luciferase transfection assay at time points of 48 and 72 hrs for compound 7N (i.e. compound 7 nM), compound 9N, linear-polyethylenimine (L-PEI) and lipofectamine (Lipo). For each compound, the 48 h timepoint is on the left and the 72 h timepoint is on the right.

FIG. 2 depicts the structures of DOPE and DOTAP.

FIG. 3 shows the transfection efficacy of cationic AM-lipid (1:10) complexes at 48 hrs, 72 hrs, 96 hrs. For each complex depicted (7N (i.e. compound 7 nM), and 9N), the bar graphs from left to right are N/P=10, N/P=25, N/P=50, and lipofectamine, respectively.

FIG. 4 shows the cytotoxicity of cationic-AMs and L-PEI to U87 glioma cells after 72 hours of exposure. Data represent mean±standard deviation (n=4). Astericks represent concentrations at which cationic-AMs elicited a significantly lower cytotoxicity than L-PEI (p<0.05).

FIG. 5 shows the characterization of AM/siRNA complexes by gel electrophoresis (A and B), dynamic light scattering (C), and zeta potential (D). Relative SybrGreen fluorescence corresponds to unbound siRNA detected in an electrophoresis band normalized by a band of free siRNA (B). Images of gel electrophoresis bands are shown with the N/P ratio for each band denoted above the band, where the mass of polymer 1 added was equivalent to 1N at the indicated N/P ratios (A). Gel electrophoresis siRNA complexation studies were performed at least three times for each polymer, and one representative gel image and band quantification is shown here. The zeta potential measurements of each polymer were compared to each other at varying N/P ratios, and asterisks in D indicate polymers whose zeta potentials differed as a function of N/P ratio (p<0.05).

FIG. 6 shows luciferase silencing in U87-Luc cells 24 hours post-transfection (A). L-PEI was used at N/P=15. The samples 5NScr and 9NScr indicate treatments with a scrambled siRNA sequence. Time-course and dose titrations were performed of 9N/siRNA complexes to U87-Luc cells (B and C). Data represent mean±standard deviation (n=3). Asterisks indicate treatments that elicited statistically significant luciferase silencing compared to the untreated control, B (A) or treatments that elicited statistically different luciferase silencing compared to the other treatment groups in the experiment (B and C) (p<0.05).

### DETAILED DESCRIPTION

Specific values listed below for radicals, substituents, and ranges, are for illustration only; they do not exclude other defined values or other values within defined ranges for the radicals and substituents. The term “alkyl” includes both straight and branched alkyls.

In one embodiment, the compound of formula I is a compound of formula I′:

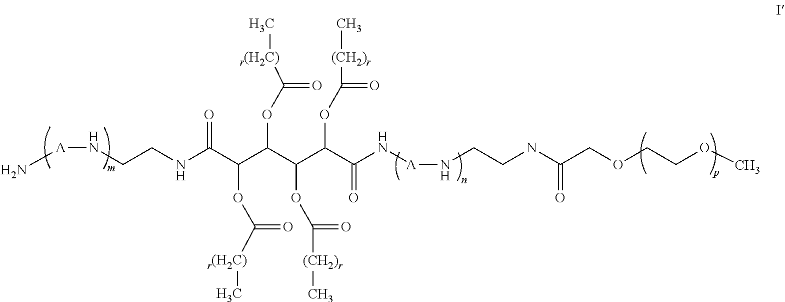

wherein:

each A is independently an ethylene group that is optionally substituted with one or more (C₁-C₃)alkyl groups;

m is an integer from 0 to 500;

n is an integer from 0 to 500;

p is an integer from 1 to 500; and

each r is independently an integer from 0 to 20;

or a salt thereof.

In one embodiment the compound of formula I is not a compound of the following formula:

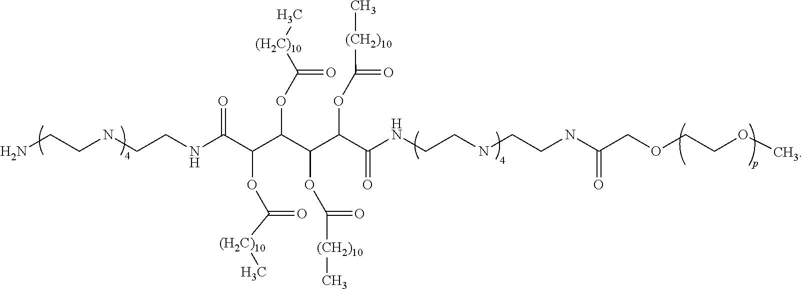

In one embodiment the compound of formula (I) is a compound of formula (Ia):

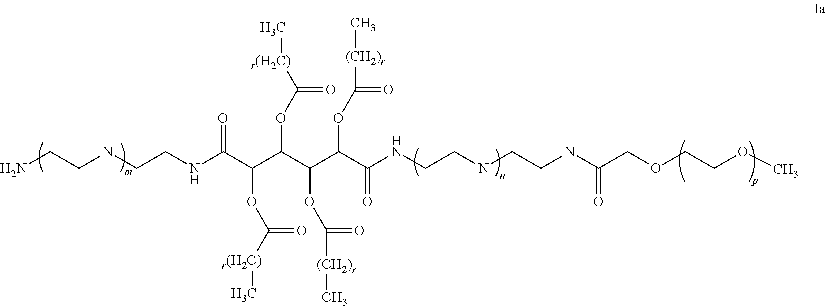

wherein: m is an integer from 0 to 500;

n is an integer from 0 to 500;

p is an integer from 1 to 500; and

each r is independently an integer from 0 to 20; or a salt thereof.

In one embodiment, the compound of formula I is a compound of formula

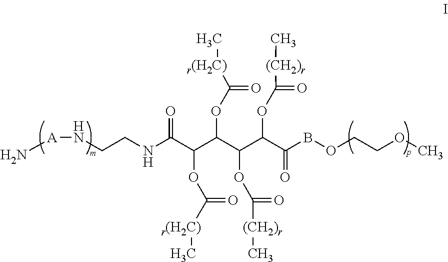

wherein:

B is absent or the group —NH(-A-NH—)ₙ(CH₂)₂—NH—C(═O)—CH₂—;

each A is independently an ethylene group that is optionally substituted with one or more (C₁-C₃)alkyl groups;

m is an integer from 0 to 500;

n is an integer from 0 to 500;

p is an integer from 1 to 500; and

each r is independently an integer from 0 to 20;

or a salt thereof.

In one embodiment m is an integer from 1 to 200.

In one embodiment m is an integer from 1 to 100.

In one embodiment m is an integer from 1 to 20.

In one embodiment m is 1, 2, 3, or 4.

In one embodiment m is 3.

In one embodiment n is an integer from 1 to 200.

In one embodiment n is an integer from 1 to 100.

In one embodiment n is an integer from 1 to 20.

In one embodiment n is 1, 2, 3, or 4.

In one embodiment n is 3.

In one embodiment p is an integer from 20 to 200.

In one embodiment p is an integer from 20 to 100.

In one embodiment p is an integer from 40 to 80.

In one embodiment p is about 109.

In one embodiment each r is independently an integer from 0 to 20.

In one embodiment each r is independently an integer from 1 to 20.

In one embodiment each r is independently 6, 7, 8, 9, 10, 11, 12, 13, 14, or 15.

In one embodiment each r is 8, 9, 10, 11, or 12.

In one embodiment each r is 10.

In one embodiment m is 2, 3, 4, or 5; n is 2, 3, 4, or 5; p is an integer from about 95 to about 125; and each r is independently 6, 7, 8, 9, 10, 11, 12, 13, 14, or 15.

In one embodiment the compound of formula (I) is a compound of the following formula (Ib):

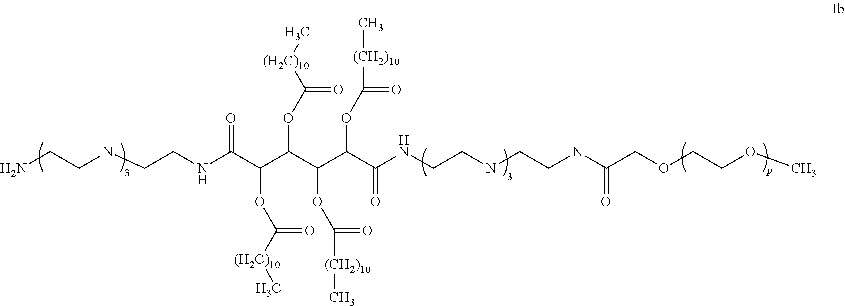

or a salt thereof.

In one embodiment for a compound of formula (Ib), p is an integer from about 20 to about 200.

In one embodiment one embodiment for a compound of formula (Ib), p is about 109.

In one embodiment the compound of the invention is a compound of the following formula:

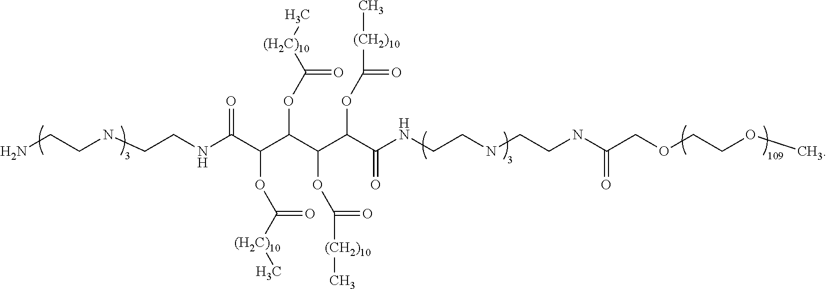

or a salt thereof.

In one embodiment the compound of the invention is a compound selected from:

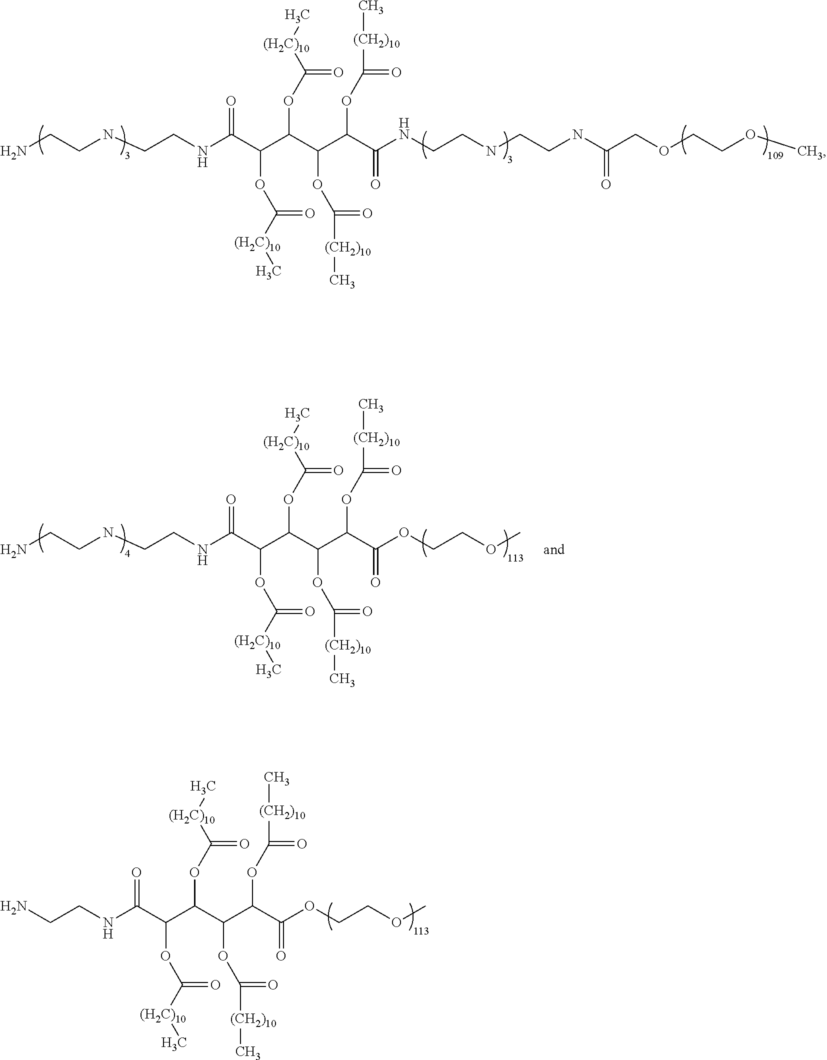

or a salt thereof.

In one embodiment the compound of formula I is not a compound of formula:

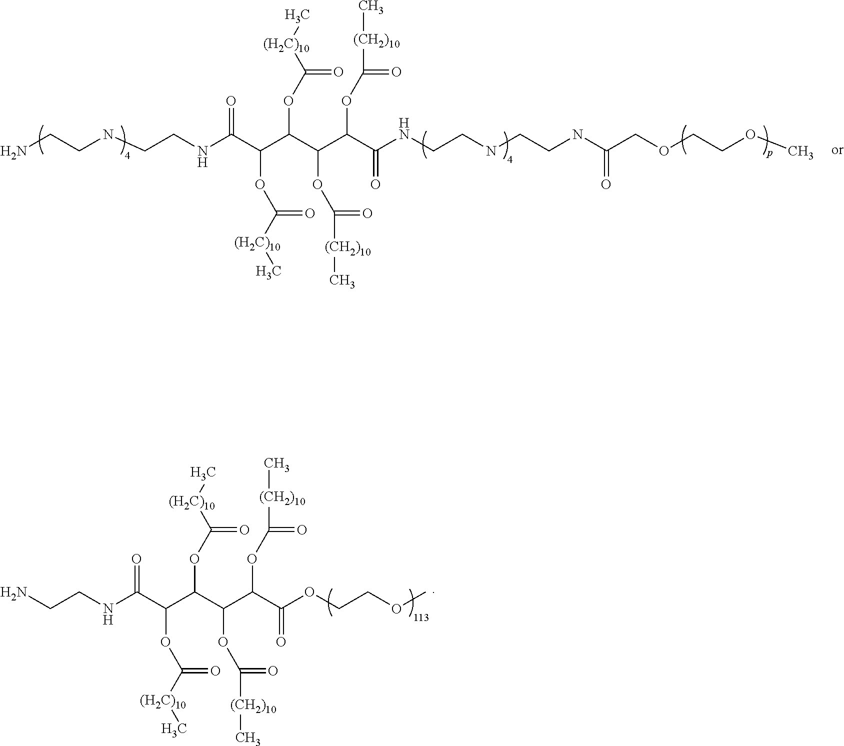

In one embodiment the compound of formula I is not a compound of formula:

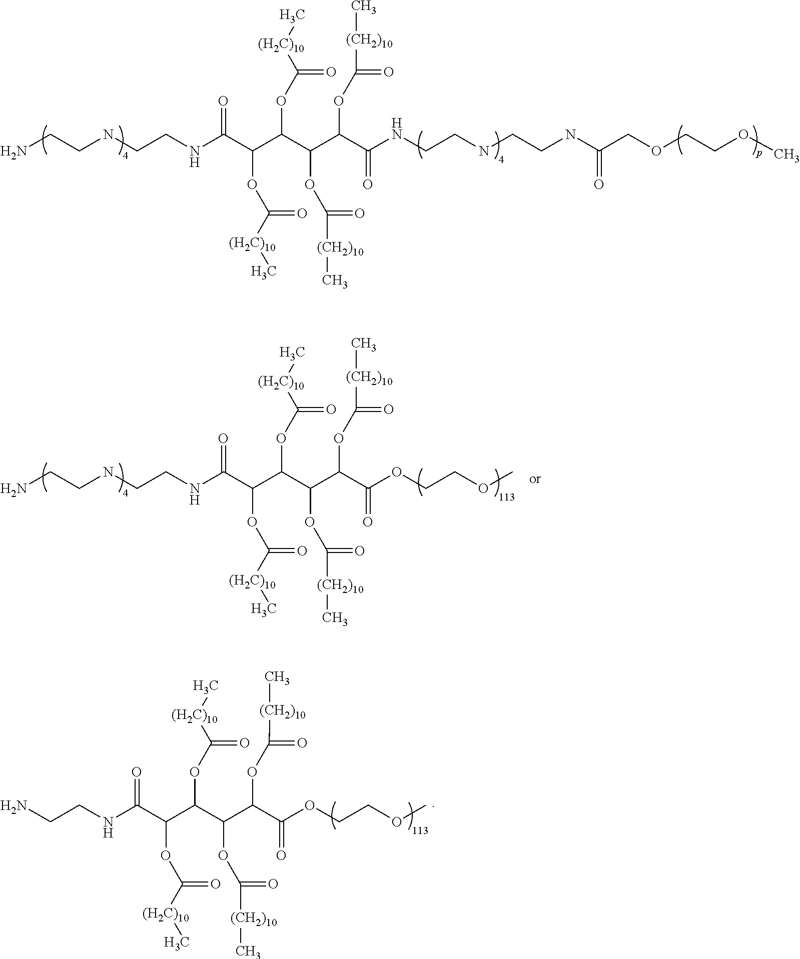

In one embodiment the compound of formula I is not a compound of formula:

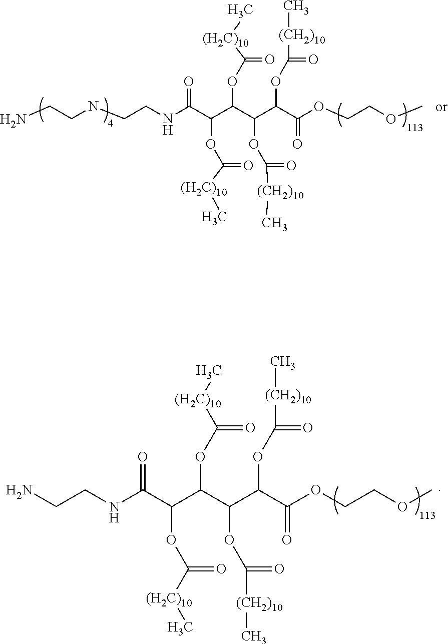

In one embodiment the compound of formula I is not a compound of formula:

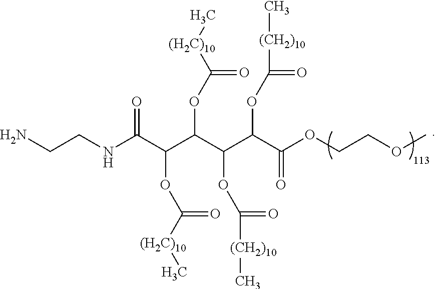

In one embodiment the compound of formula I is not a compound of formula:

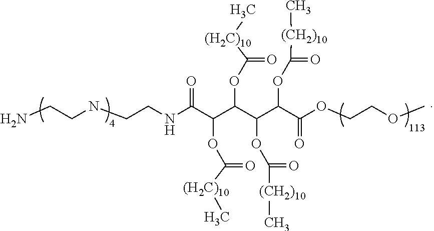

In one embodiment the value for B is absent (i.e. the carbonyl adjacent to B and the oxygen adjacent to B are connected (for the compound of formula I)).

In one embodiment the value for B is the group —NH(-A-NH—)ₙ—(CH₂)₂—NH—C(═O)—CH₂—.

It is to be understood that the group —NH(-A-NH—)ₙ—(CH₂)₂—NH—C(═O)—CH₂— is the structure:

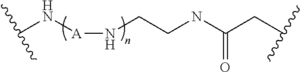

wherein the wavy lines represent points of attachment to the compound of formula I.

It also to be understood that when the group —NH(-A-NH—)ₙ—(CH₂)₂—NH—C(═O)—CH₂— for B is included in the compound of formula I the group is incorporated into the compound of formula I as drawn (e.g. the —NH part of the B group is on the left hand side and bonded to the carbonyl of the compound of formula I and the CH₂— part of the B group is on the right and side and bonded to the oxygen atom of the PEG group of the compound of formula I).

In one embodiment the nucleic acid is DNA, RNA or a siRNA molecule.

In one embodiment the nucleic acid is DNA.

In one embodiment the nucleic acid is RNA.

In one embodiment the nucleic acid is a siRNA molecule.

A compound of formula I can be prepared using procedures similar to those described in L. Tian, L. Yam, N. Zhou, H. Tat, and K. Uhrich, Macromolecules 2004, 37, 538, or using the procedures described herein.

The term “alkyl” includes both straight and branched hydrocarbon groups.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The term “nucleotide sequence” refers to a polymer of DNA or RNA which can be single-stranded or double-stranded, optionally containing synthetic, non-natural or altered nucleotide bases capable of incorporation into DNA or RNA polymers. The terms “nucleic acid,” “nucleic acid molecule,” or “polynucleotide” are used interchangeably.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The following terms are used to describe the sequence relationships between two or more nucleotide sequences: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm. Non-limiting examples of such mathematical algorithms are the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, J M B, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)) are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present invention, comparison of nucleotide sequences for determination of percent sequence identity to the promoter sequences disclosed herein may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, J M B, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, certain embodiments of the invention provide nucleic acid molecules that are substantially identical to the nucleic acid molecules described herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl (1984); Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration is increased so that a higher temperature can be used. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. For short nucleotide sequences (e.g., about 10 to 50 nucleotides), stringent conditions typically involve salt concentrations of less than about 1.5 M, less than about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids that have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

In addition to the chemical optimization of stringency conditions, analytical models and algorithms can be applied to hybridization data-sets (e.g. microarray data) to improve stringency.

The present invention further provides a method of substantially silencing a target gene of interest or targeted allele for the gene of interest in order to provide a biological or therapeutic effect. As used herein the term “substantially silencing” or “substantially silenced” refers to decreasing, reducing, or inhibiting the expression of the target gene or target allele by at least about 5%, 10%, 15%, 20%, 25%, 30%, 35%, 40%, 45%, 50%, 55%, 60%, 65%, 70%, 75%, 80%, 85% to 100%. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the disease. Therapeutic effects can be measured quantitatively by a physician or qualitatively by a patient afflicted with the disease state targeted by the siRNA. In certain embodiments wherein both the mutant and wild type allele are substantially silenced, the term therapeutic effect defines a condition in which silencing of the wild type allele's expression does not have a deleterious or harmful effect on normal functions such that the patient would not have a therapeutic effect. As used herein, the term “biological effect” refers to a change in the behavior of a cell, tissue or organism. Biological effects encompass a wide range of behaviors that include but are not limited to changes in gene expression, metabolism, growth, motility, or response to environmental perturbations. Biological effects can be measured by a qualified scientist or technician using assays specific to the biological effect under study.

An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, huntingtin. As used herein, the term “siRNA” is a generic term that encompasses the subset of shRNAs and miRNAs. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding ataxin-1 or huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compositions of the invention (e.g. compounds of formula I and a nucleic acid) can be formulated as pharmaceutical compositions and administered to a mammalian host, such as a human patient in a variety of forms adapted to the chosen route of administration, i.e. orally, parenterally, by intravenous, intramuscular, topical or subcutaneous routes. Thus, the compositions of the invention may be systemically administered, in combination with a pharmaceutically acceptable vehicle such as an inert diluent.

The compositions of the invention may be administered intravenously or intraperitoneally by infusion or injection. Solutions of the compositions can be prepared, for example, in water. Under ordinary conditions of storage and use, these preparations may contain a preservative to prevent the growth of microorganisms.

The pharmaceutical dosage forms suitable for injection or infusion should be sterile, fluid and stable under the conditions of manufacture and storage. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the encapsulates of the invention in the required amount in the appropriate solvent with various of the other ingredients enumerated above, as required, followed by sterilization.

The dose and method of administration will vary from animal to animal and be dependent upon such factors as the type of animal being treated, its sex, weight, diet, concurrent medication, overall clinical condition, the particular therapeutic agent employed, the specific use for which the agent is employed, and other factors which those skilled in the relevant field will recognize.

Therapeutically effective dosages may be determined by either in vitro or in vivo methods. For each particular dosage form of the present invention, individual determinations may be made to determine the optimal dosage required. The range of therapeutically effective dosages will naturally be influenced by the route of administration, the therapeutic objectives, and the condition of the patient. The determination of effective dosage levels, that is, the dosage levels necessary to achieve the desired result, will be within the ambit of one skilled in the art. Typically, applications of agent are commenced at lower dosage levels, with dosage levels being increased until the desired effect is achieved.

The invention also includes complexes (e.g. liposomes) comprising a compound of formula I or a salt thereof and one or more lipids. Such AM/lipid complexes and compositions thereof are useful for the delivery of nucleic acids (e.g. DNA, RNA or siRNA).

Useful lipids include-1,2-dioleoyl-sn-glycero-3-phosphoethanolamine (DOPE) and 1,2-dioleoyl-3-trimethylammonium-propane (DOTAP). Other lipids include, but at not limited to, N-[1-(2,3-dioleyloxy) propyl]-N,N,N-tri-methylammoniumchloride (DOTMA), dioleoyl phosphatidyl choline (DOPC), dioctadecylamidoglicylspermin (DOGS), 2,3-dioleoyloxy-N-[2-(spermine-carboxamido)ethyl]-N,N-dimethyl-1-propanaminum (DOSPA), 1,2-distearoyl-3-dimethylammonium-propane (DODAP), 1,2-Bis(diphenylphosphino)ethane (dppe), Dimethyldioctadecylammonium-Bromide Salt (DDAB). Theses lipids can be used alone, or as a mixture with one or more lipids. The mass ratio of the mixture can be equal or unequal.

The AM/lipid complexes can be formed from a compound of formula I and one or more lipids. The complexes can include different ratios of lipids. For example, when two lipids are present the weight ratios of the two lipids to one another can be 9:1, 4:1, 7:3, 3:2 or 1:1 or any useful ratio to obtain the desired properties. The ratio of the cationic AMs (i.e. compounds of formula I) to the lipids can also be varied. For example, the weight ratios of the AMs to lipids can be 0:1, 1:10, 1:5, 1:2, 1:1, 2:1, 5:1, 10:1 and 1:0.

The invention will now be illustrated by the following non-limiting Examples.

### Example 1

### A Cationic Polymer for Nucleic Acid Delivery

A representative compound (polymer) of the invention was prepared and evaluated as a nucleic acid delivery vehicle by measuring its hydrodynamic diameter and its zeta potential.

The compound 7 nM was synthesized as shown in Scheme A3.1. In this synthetic method, the formation of oligomers is controlled by stoichiometry and the slow addition of the starting materials via the syringe pump.

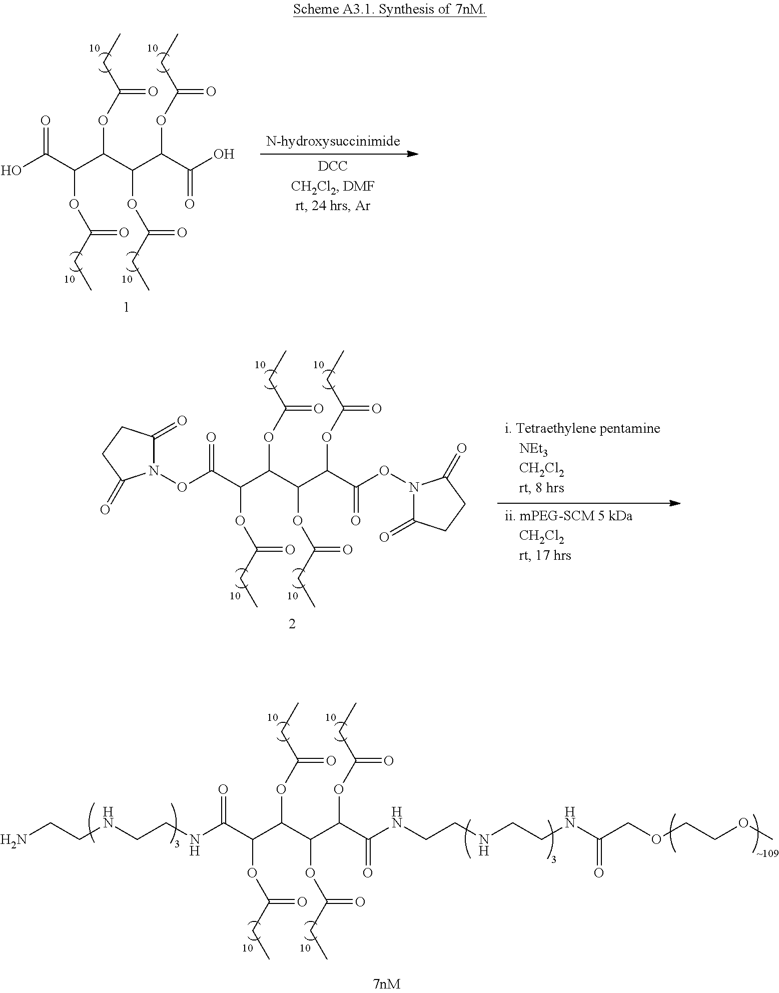

Successful synthesis of the polymer was verified by proton nuclear magnetic resonance (¹H NMR) spectroscopy and the molecular weight was determined by gel permeation chromatography (GPC) relative to PEG standards. Due to the abundance of PEG in the polymer (˜83% of protons), the presence of new protons in the ¹H NMR spectra were difficult to detect, particularly from 0.8 to 2.4 ppm where the methylene protons of the hydrophobic chains comprise the majority of that region. Thus, the spectra were monitored for the disappearance of the protons of the activating group (N-hydroxysuccinimide) on 2, which resonate at 2.8 ppm. In addition, a new, broad peak resonating at ˜2.30 ppm for the ethylene spacer indicated successful conjugation of the tetraethylene pentamine to the polymer. However, integrations indicated that there was a mixture of di-PEGylated polymer and mono-PEGylated polymer (i.e. 2 PEGs to 4 alkylated arms).

The polymer was further characterized by dynamic light scattering for its ability to form micelles and by zeta potential to confirm a cationic polymer was indeed obtained. The hydrodynamic diameter was determined to be 92.2±39.4 nm while the zeta potential was 30.7±2.9 mV. The large positive magnitude of the zeta potential suggests that this polymer is a good candidate for delivering nucleic acids (e.g. siRNA).

### Example 2

### Alternative Route to Compound 7 nM

In Example 1, the only control over the formation of oligomers is the stoichiometry and the slow addition of the starting materials using the syringe pump. To better control the synthesis of these polymers and isolate a more pure product, two protection schemes were designed and evaluated.

To control the final structure of 7 nM, a protection step was added to the synthetic procedure. As a model synthesis, only 7 nM was evaluated. The protection step, the addition of benzyl chloroformate (CBZ-C1) was added following the addition of tetraethylene pentamine to 2, as shown in Scheme A3.2. It should be noted that an effort was made to isolate and purify 3, but once it was isolated as a white solid, it was insoluble in further solvents for chemical characterization. Thus, in the final synthesis, it was not isolated. The CBZ protecting group was removed by hydrogenation in the final step of the synthesis.

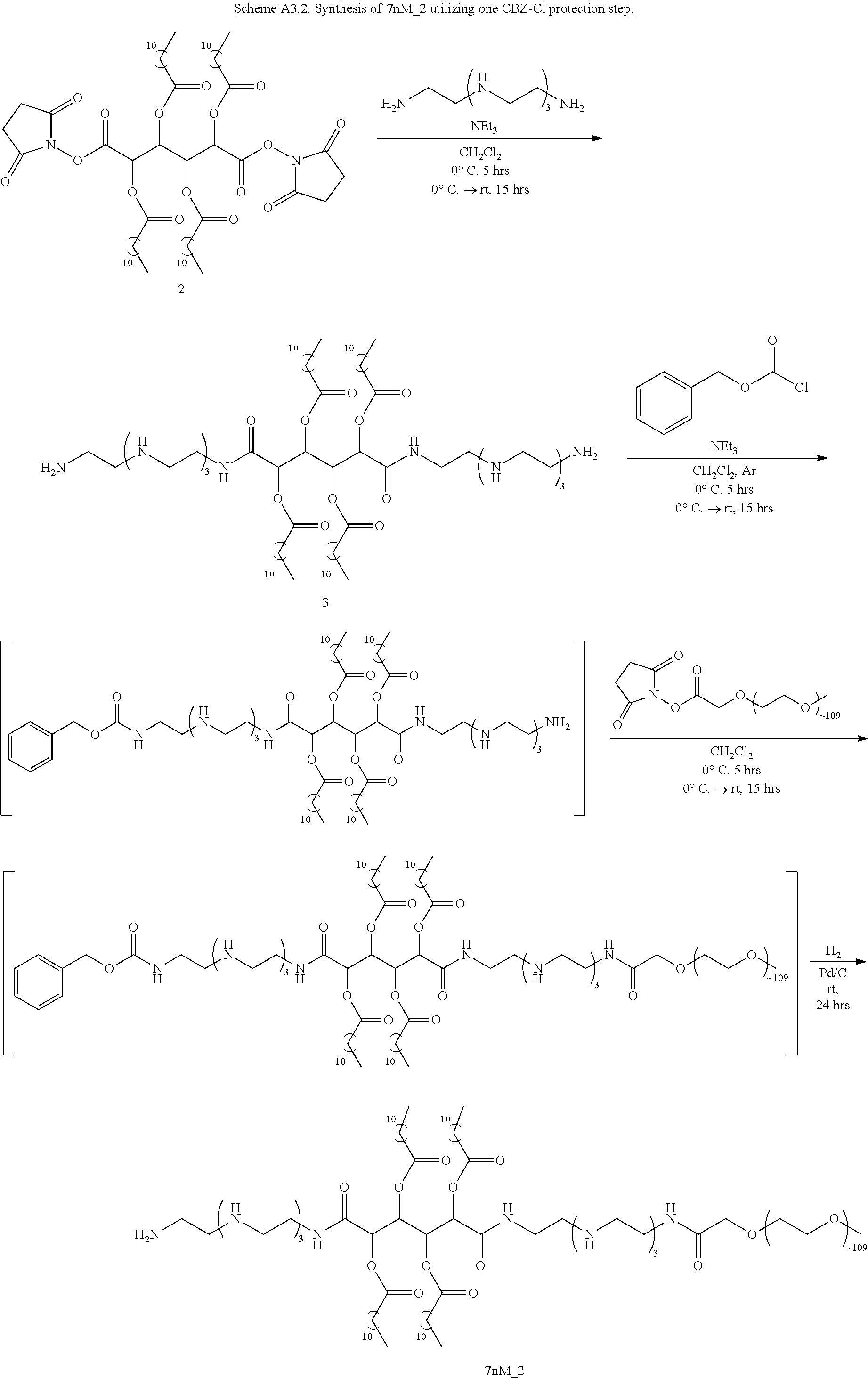

In comparison to the 7 nM synthesized in Example 1, the ¹H NMR of 7 nM_{—}2 from Example 2 had several differences. Specifically, in the spectra for 7 nM, the broad peak at 2.30 ppm was assigned to the ethylenes of the tetraethylene pentamine groups. However, in the spectra for 7 nM_{—}2 from Example 2, two new, distinct peaks were observed and assigned to the ethylenes of the tetraethylene pentamine groups and the hydrogens of the alkylated arms on the mucic acid derivative, which are shifted downfield due to the amino groups. The first was a quartet at 3.12 ppm integrating for ˜22 protons and the second a quartet at ˜1.42 ppm integrating for ˜32 protons. The number of protons is due to overlap of the ethylenes of the tetraethylene pentamine groups with some methylenes of the alkylated arms, which shift downfield due to interactions with the electronegative amino groups (the peak that normally exists at ˜2.4 ppm integrating for 8 protons is no longer there). In addition, a broad peak at 12.2 ppm integrating for 7 protons was assigned the amino hydrogens.

The polymer was further characterized by dynamic light scattering for its ability to form micelles and by zeta potential to confirm a cationic polymer was indeed obtained. The hydrodynamic diameter was determined to be 174.4±42.5 nm while the zeta potential was 24.9±3.1 mV.

Thus, this synthetic procedure appears to be a viable method for preparing compound 7 nM.

### Example 3

### Evaluation of the siRNA Delivery Efficacy of Compound 7N (Compound 7N is the Same Compound as Compound 7 nM) and 9N (Compound 9N is Described in Example 5)

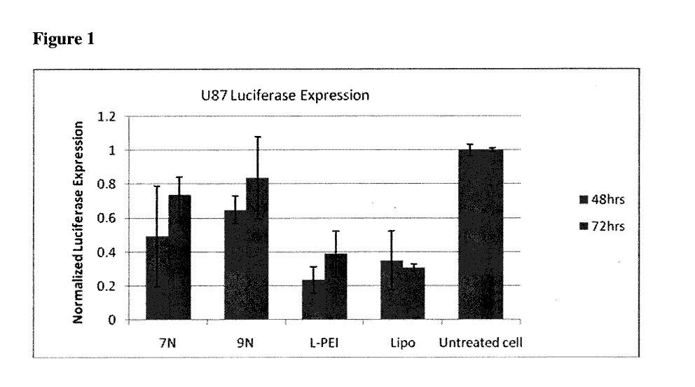

The siRNA delivery efficacy of the cationic AM complexes was tested using a human primary glioblastoma cell line (U87 MG). By loading the cationoic AMs complexes with anti-luciferase siRNA at N/P ratio of 50, knockdown of luciferase expression was achieved and quantified at 48 hrs and 72 hrs. FIG. 1 shows the knockdown efficacy of compounds 7N and 9N compared to linear-polyethylenimine (L-PEI) and lipofectamine (Lipo). It has been demonstrated that cationic AMs have lower cytotoxicity than the L-PEI and lipofectramine

### Example 4

### Preparation and Evaluation of AM-Lipid Complexes

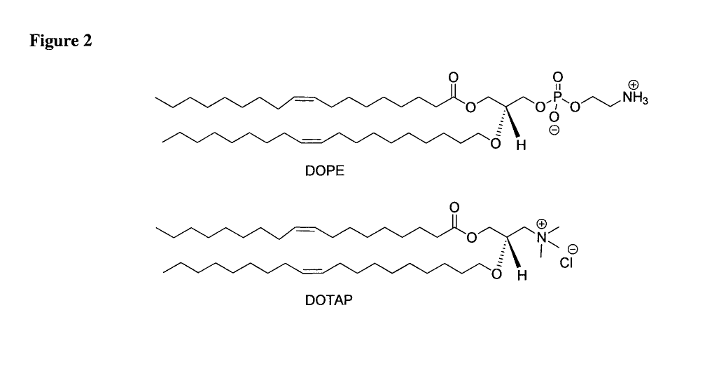

AM-lipid complexes were prepared by incorporating cationic AMs compound 7N (i.e. compound 7 nM) or compound 9N into liposomes composed of 1,2-dioleoyl-sn-glycero-3-phosphoethanolamine (DOPE) and 1,2-dioleoyl-3-trimethylammonium-propane (DOTAP) at a 1:1 weight ratio of DOPE:DOTAP (FIG. 2). The cationic AMs were incorporated into the DOPE:DOTAP liposomes at various AM:lipid ratios using well-established methods (Harmon, A. M., et al., Preferential cellular uptake of amphiphilic macromolecule-lipid complexes with enhanced stability and biocompatibility, Journal of Controlled Release, 2011. 153(3): p. 233-239).

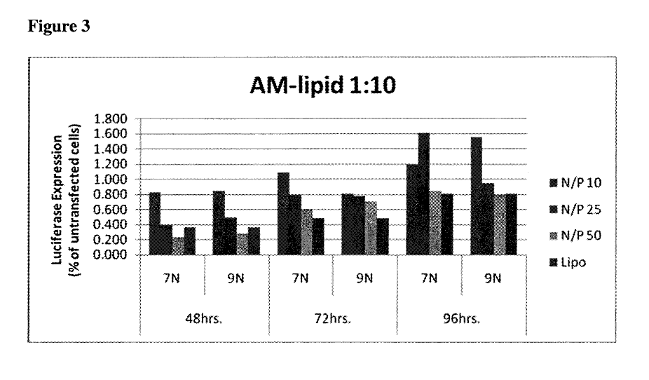

The transfection efficacy of cationic AM-lipid complexes was evaluated by assays in U87 cell line using lipofectamine as positive control. The cationic AM:lipid weight ratios assayed were 0:1, 1:10, 1:5, 1:2, 1:1, 2:1, 5:1, 10:1 and 1:0 using previously reported methods, co-evaporation and post-addition (Harmon, A. M., et al., Preferential cellular uptake of amphiphilic macromolecule-lipid complexes with enhanced stability and biocompatibility, Journal of Controlled Release, 2011. 153(3): p. 233-239). For the co-evaporation method, cationic AMs and lipid were co-dispensing aliquots in chloroform into glass vial and solvent removed by rotary evaporation. The remaining films were hydrated with 10 mM 4-(2-hydroxyethyl)-1-piperazineethanesulfonic acid (HEPES) buffer overnight at room temperature. The materials were then extruded 21 times through a mini-extruder to give unilamellar liposomes. For the post-addition method, cationic AM aliquots in 10 mM HEPES were added to extruded DOPE:DOTAP liposomes. The formed cationic AM lipid complexes were then complexed with anti-luciferase siRNA at N/P ratios of 10, 25, and 50. The transfection in the U87 MG cell line was performed at time points of 48, 72, and, 96 hrs using lipofectamine as the positive control. For cationic AM-lipid ratio of 1:10 shown in FIG. 3, 7N and 9N complexes with lipids showed comparable or slightly better efficacy than lipofectamine.

Certain embodiments of the invention are exemplified in the document “S. M. Sparks, C. L. Waite, A. M. Harmon, L. M. Nusblat, C. M. Roth, K. E. Uhrich, Efficient Intracellular siRNA Delivery by Ethyleneimine-Modified Amphiphilic Macromolecules. Macromol. Biosci., 2011, 11, 1192-1200” which is incorporated by reference in its entirety. Example 5 below describes some of these embodiments.

### Example 5

### Preparation and Physical/Biological Evaluation of Polymers 9N, 1N and 5N (the Compound Numbers Used in this Example Correspond to those Shown in Scheme B Below)

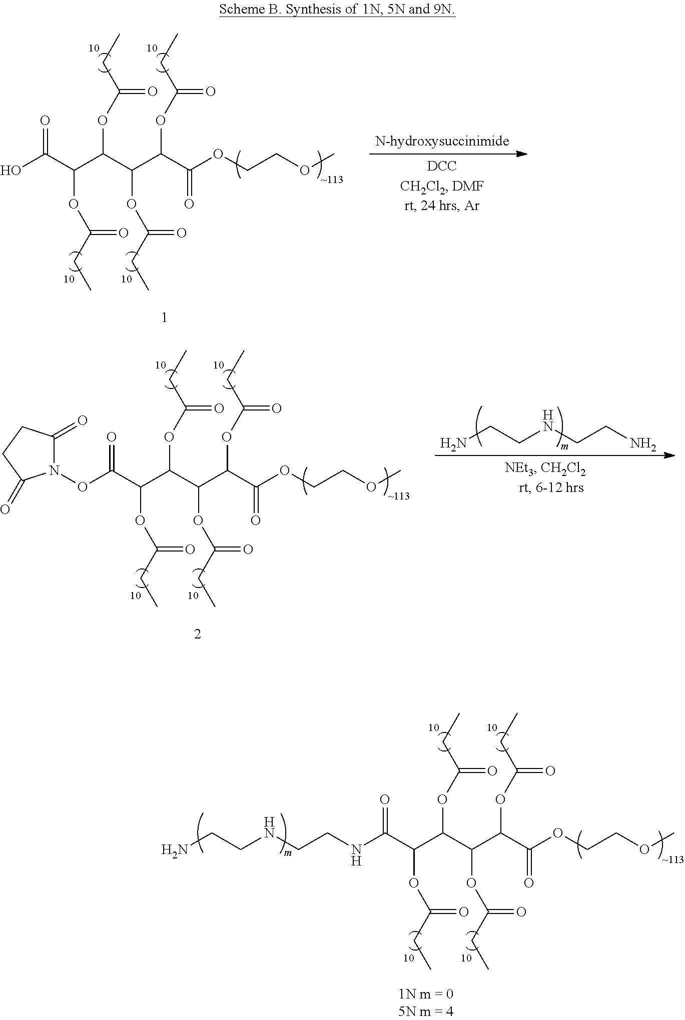

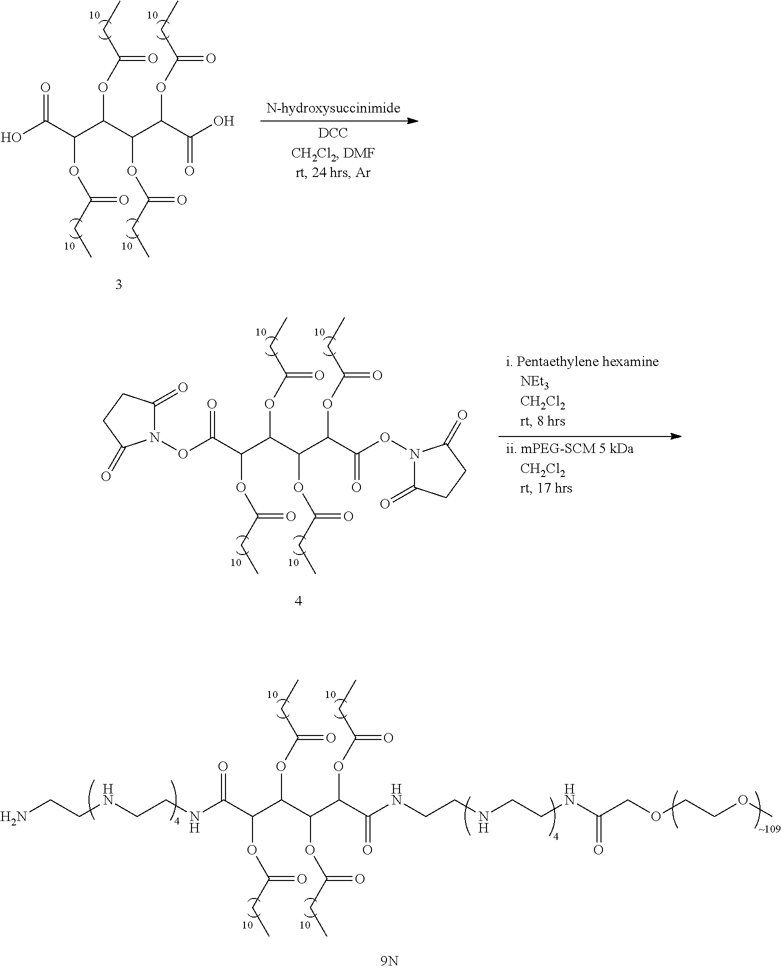

### Synthetic Materials:

Unless otherwise stated, solvents and reagents were purchased from Fisher Scientific (Pittsburgh, Pa.) and Sigma-Aldrich (St. Louis, Mo.) and used as received. Poly(ethylene glycol) 5 kDa was purchased from Polysciences, Inc. (Warrington, Pa.) and dried by azeotropic distillation from toluene before use. N-hydroxysuccinimide(NHS)-functionalized PEG, Methoxy-PEG-succinimidyl carboxymethyl (MW 5 kDa) (mPEG-SCM). was purchased from Laysan Bio, Inc (Arab, A L) and used as received. Compounds 1, 2, and 3 were prepared as previously described (L. Tao, K. Uhrich, Journal of Colloid and Interface Science 2006, 298, 102; J. Djordjevic, M. Barch, K. Uhrich, Pharmaceutical Research 2005, 22, 24.)

### Polymer Characterization Methods:

Proton nuclear magnetic resonance (¹H-NMR) spectra of the products were obtained using a Varian 400 MHz or 500 MHz spectrophotometer. Samples were dissolved in chloroform-d, with a few drops of dimethyl sulfoxide-d₆ if necessary, with tetramethylsilane as an internal reference. Molecular weights (Mw) and polydispersity indices (PDI) were determined using gel permeation chromatography (GPC) with respect to PEG (Sigma-Aldrich) on a Waters Stryagel® HR 3 THF column (7.8×300 mm). The Waters LC system (Milford, Mass.) was equipped with a 2414 refractive index detector, a 1515 isocratic HPLC pump, and 717plus autosampler. An IBM ThinkCentre computer with Waters Breeze Version 3.30 software installed was used for collection and processing of data. Samples were prepared at a concentration of 10 mg/mL in tetrahydrofuran, filtered using 0.45 μm pore size nylon or polytetrafluoroethylene syringe filters (Fisher Scientific) and placed in sample vials to be injected into the system. Melting points were determined by differential scanning calorimetry (DSC) on a TA DSC Q200. TA Universal Analysis 2000 software was used for data collection on a Dell Dimension 3000 computer. Samples (3-5 mg) were heated under dry nitrogen gas. Data were collected at heating and cooling rates of 10° C./min with a two-cycle minimum.

### Polymer Synthesis:

1N: Ethylenediamine (50 μL, 0.75 mmol) was dissolved in HPLC-grade CH₂Cl₂ (3 mL) and triethylamine (0.15 mL, 1.1 mmol). In a separate vessel, 2 (0.51 g, 0.085 mmol) was dissolved in HPLC-grade CH₂Cl₂ (9 mL) and subsequently added to the solution of ethylenediamine dropwise via syringe pump at a rate of 1.0 mL/hr. The reaction was stirred overnight (˜18 hrs). The reaction solution was then diluted with CH₂Cl₂ and subsequently washed with 0.1 NHCl/brine (1×) and brine (2×). The combined aqueous portions were extracted with CH₂Cl₂ and the combined organics dried over MgSO₄, and concentrated to a yellow oil. The desired product was precipitated from the oil dissolved in CH₂Cl₂ (5 mL) by addition of 10-fold diethyl ether and cooling over dry ice for 1 hr. The solid was then collected by centrifugation at 3000 rpm for 5 min and the supernatant removed by decanting. The resulting white solid was dried under ambient atmosphere (12 hrs) and under high vacuum (12 hrs). Yield: 0.41 g, 80%. ¹H-NMR (CDCl₃): δ 5.67 (m, 2H, CH), 5.14 (m, 2H, CH), 4.24 (m, 3H, CH₂), 3.60 (m, ˜0.45 kH, CH₂O), 3.37 (s, 3H, OCH₃), 2.37 (m, 8H, CH₂), 2.29 (m, 4H, CH₂), 1.81 (b, 4H, CH₂), 1.60 (m, 8H, CH₂), 1.26 (m, 64H, CH₂), 0.87 (t, 12H, CH₃). Tₘ=58° C. GPC: M_{w}: 6.3 kDa; PDI: 1.1.

5N: Pentaethylenehexamine (0.15 mL, 0.64 mmol) was dissolved in HPLC-grade CH₂Cl₂ (10 mL) and triethylamine (0.33 mL, 2.4 mmol). In a separate vessel, 2 (0.48 g, 0.079 mmol) was dissolved in HPLC-grade CH₂Cl₂ (10 mL) and subsequently added to the solution of ethylenediamine dropwise via syringe pump at a rate of 1.0 mL/hr. The reaction was stirred overnight (˜17 hrs). The bright yellow reaction solution was diluted with CH₂Cl₂ and subsequently washed with 0.1 N HCl/brine (1×) and brine (2×). The combined aqueous portions were extracted with CH₂Cl₂ and the combined organics dried over MgSO₄, and concentrated to a cloudy yellow oil. The desired product was precipitated from the oil dissolved in CH₂Cl₂ (5 mL) by addition of 10-fold diethyl ether and cooling over dry ice for 1 hr. The solid was then collected by centrifugation at 3000 rpm for 5 min and the supernatant removed by decanting. The resulting white solid was dried under ambient atmosphere (12 hrs) and under high vacuum (12 hrs). Yield: 0.42 g, 86%. ¹H-NMR (DMSO): δ 5.50 (m, 2H, CH), 5.11 (m, 2H, CH), 3.41 (m, ˜0.45 kH, CH₂O), 3.24 (s, 3H, OCH₃), 2.89 (m, 13H, CH₂), 2.80 (bs, 2H, CH₂), 2.76 (bs, 7H, CH₂), 2.64 (bs, 6H, CH₂), 1.49 (m, 8H, CH₂), 1.24 (m, 64H, CH₂), 0.84 (t, 12H, CH₃). Tₘ=59° C. GPC: M_{w}: 6.4 kDa; PDI: 1.1.

4: Product 3 (5.10 g, 5.43 mmol) and NHS (5.38 g, 46.8 mmol) were dissolved in anhydrous CH₂Cl₂ (100 mL) and anhydrous DMF (18 mL) under argon. Once a clear solution was obtained, N,N′-dicyclohexylcarbodiimide (17 mL, 17 mmol) was added and the reaction stirred at room temp under argon for 24 hours. The resulting solution with white suspension was stored at −4° C. overnight. The dicyclohexyl urea (DCU) byproduct was then removed by vacuum filtration and the filtrate washed with 0.1 N HCl and 50:50 brine/H₂O, dried over MgSO₄, and concentrated. The resulting white solid was then dissolved in a small amount of CH₂Cl₂ (5-10 mL) and stored at −4° C. for 2-3 hours. The resulting white suspension was filtered to remove residual DCU. The filtrate was then concentrated to dryness and the white solid dried under high vacuum overnight. Yield=4.5 g, 73%. ¹H-NMR (CDCl₃): δ=5.96 (s, 2H, CH), 5.57 (s, 1H, CH), 2.81 (s, 8H, CH₂), 2.49 (m, 6H, CH₂), 2.37 (m, 2H, CH₂), 1.64 (m, 8H, CH₂), 1.27 (m, 64H, CH₂), 0.89 (t, 12H, CH₃).

9N: Pentaethylenehexamine (0.05 mL, 0.2 mmol) was dissolved in CH₂Cl₂ (3 mL) and triethylamine (0.15 mL, 1.1 mmol). In a separate vessel, 4 (0.10 g, 0.090 mmol) was dissolved in HPLC-grade CH₂Cl₂ (3 mL) and subsequently added to the solution of ethylenediamine dropwise via syringe pump at a rate of 1.0 mL/hr. The reaction was stirred at room temperature a total of 8 hrs. mPEG-SCM (0.45 g, 0.090 mmol) dissolved in CH₂Cl₂ (7 mL) was then added to the yellow reaction solution dropwise via syringe pump at a rate of 1.0 mL/hr. The reaction was stirred at room temperature overnight (˜17 hrs). The solvent was then removed from the reaction solution by rotary evaporation. The oil/solid was then redispersed in CH₂Cl₂ and filtered to remove the solid NHS-byproduct. The filtrate was concentrated to an oil and product precipitated from the oil dissolved in CH₂Cl₂ (5 mL) by addition of 10-fold diethyl ether. The solid was then collected by centrifugation at 3000 rpm for 5 min and the supernatant removed by decanting. The resulting white solid was washed with diethyl ether (1×) and dried under ambient atmosphere (12 hrs) and under high vacuum (12 hrs). Yield: 0.45 g, 87%. ¹H-NMR (CDCl₃): δ 7.26 (s, 4H, CH), 3.69 (m, ˜0.44 kH, CH₂O), 3.38 (s, 3H, OCH₃), 3.05 (bm, 15H, CH₂), 2.55 (bm, 16H, CH₂), 2.07 (bm, 40H, CH₂), 1.65 (bs, 7H, CH₂), 1.48 (t, 5H, CH₂), 1.26 (m, 37H, CH₂), 0.88 (t, 12H, CH₃). Tₘ=59° C. GPC: 5.5 kDa; PDI=1.1.

### Size and Zeta Potential of AMs and AM/siRNA Complexes:

Dynamic light scattering (DLS) and zeta potential analyses were performed using a Malvern Instruments Zetasizer Nano ZS-90 instrument (Southboro, Mass.). DLS measurements were performed at a 90° scattering angle at 25° C. Size distributions by volume of measurements were collected in triplicate, averaged and reported. Zeta potential measurements were collected in triplicate, averaged and the Z-average charges reported. For all measurements, error bars represent peak widths of the average value.

### Sample Preparation:

AMs alone: Polymer solutions at a concentration of 1.0 mg/mL were prepared using picopure water and filtered with a 0.45 μM Nylon syringe filter (Fischer Scientific, Pittsburgh, Pa.).

AM/siRNA complexes: Complexes were prepared in picopure water at various nitrogen/phosphate (N/P) ratios. For size and zeta potential measurements, 2 mL of solutions containing AM/siRNA complexes were prepared at polymer concentrations sufficient for detection by the zetasizer instrument (1 mg/mL for 1 and 1N, and 2 mg/mL for 5N and 9N). Solutions were briefly vortexed and incubated for at least 60 min at room temperature to allow for complex formation prior to size and zeta potential analysis.

### Gel Electrophoresis:

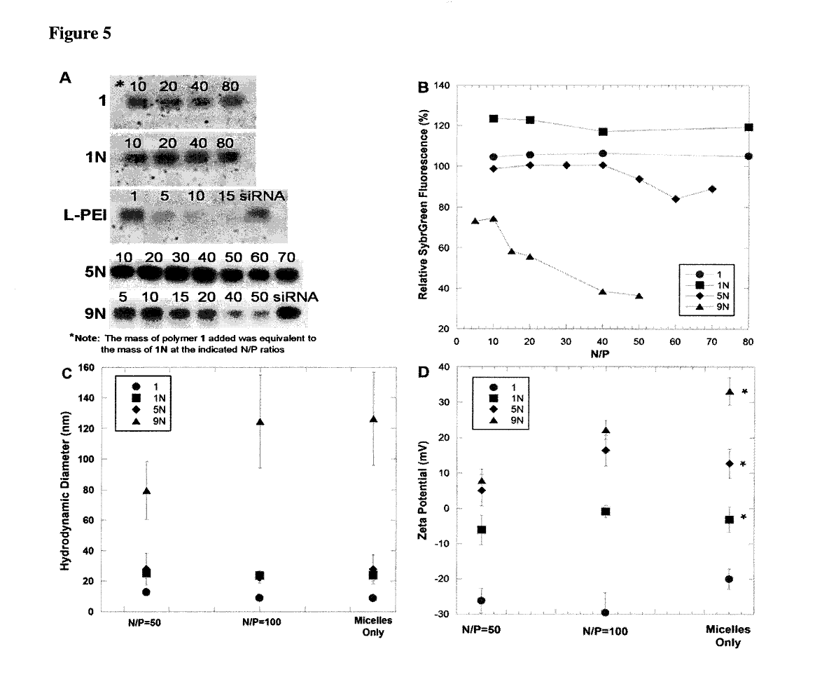

Polymer/siRNA (Dharmacon, Lafayette, Colo.) complexes were first prepared at the desired nitrogen to phosphorous (N/P) ratios by mixing solutions of polymers (stocks maintained in DI water) and siRNA in PBS (final siRNA concentration of 12.5 μg/mL). Since polymer 1 does not contain primary amines, the mass of polymer 1 added for the gel electrophoresis experiments was equivalent to the mass of polymer 1N added at the N/P ratios indicated in FIG. 5. Solutions were briefly vortexed, and incubated for 60 min at room temperature to allow for complex formation. Polymer/siRNA complexes were loaded into 1% agarose gels run in an electrophoresis chamber at 70 V for 40 minutes. Following electrophoresis, gels were stained with SYBR Green II RNA gel stain (Invitrogen, Carlsbad, Calif.) for 30 minutes prior to imaging on a Bio-Rad Molecular Imager FX (Bio-Rad Laboratories, Hercules, Calif.) to visualize unbound siRNA. The fluorescence intensities of bands were quantified using Quantity One Quantitation software (Bio-Rad Laboratories, Hercules, Calif.).

### Cell Culture:

All cell culture products were obtained from Invitrogen (Carlsbad, Calif.). U87 MG cells (ATCC HTB-14) were maintained in DMEM medium supplemented with 10% fetal bovine serum (FBS), L-glutamine, and penicillin-streptomycin. A U87 cell line containing a stably integrated destabilized EGFP (d1EGFP) transgene (U87-GFP) was generated as described previously, ^{[33]}and was maintained under constant selective pressure by G418 (500 μg/mL), and the growth medium was supplemented with sodium pyruvate and nonessential amino acids. U87-Luc, a human glioblastoma cell line with constitutive expression of firefly luciferase, was generously provided by Dr. Xu-Li Wang (Department of Pharmaceutics and Pharmaceutical Chemistry, University of Utah). U87-Luc cells were maintained in minimal essential medium supplemented with 10% FBS, penicillin-streptomycin, and maintained under selective pressure by G418.

### Cytotoxicity Assay:

U87 glioma cells were seeded into 96 well plates (Corning, Corning, N.Y.) at 10,000 cells per well in DMEM supplemented with 10% FBS and 1% penicillin-streptomycin and incubated overnight at 37° C., with 5% CO₂. The media was removed by aspiration and replaced with 200 μL aminated-AM or PEI dissolved in media at desired concentrations (n=4 per condition). Untreated control wells received media only. After 72 hours, cells were harvested by trypsinization (75 μL trypsin-EDTA followed with 75 μL complete media to neutralize trypsin) and 50 μl, of staining solution (48:1:1 media:DMSO:Guava ViaCount Flex reagent (Guava Technologies, Hayward, Calif.) was added to each well. Cells were counted using a Guava EasyCyte Plus (Guava Technologies, Haywood Calif.) instrument with an original volume of 0.2 mL and a dilution factor of one.

siRNA Delivery Assay:

U87-Luc cells were plated at a density of 5000 cells/well in 96-well plates approximately 20 hours prior to transfection. Immediately prior to transfection, polymer/siRNA complexes were prepared in 20 μl, of PBS (N/P=50 for the AMs, and N/P=15 for linear PEI). Linear polyethyleneimine (Polysciences, Inc., Warrington, Pa.) commonly used polymeric transfection reagent, was used as a positive control. An irrelevant siRNA sequence not targeted against firefly luciferase was delivered as a negative control. The polyplexes were brought to a total volume of 100 μL, in OptiMEM medium to obtain a final siRNA concentration of 100 nM. The serum-containing culture medium was aspirated from the cells, and each well treated with 100 μL of the polyplexes in OptiMEM medium. Each treatment was performed in triplicate. After a 4 hr incubation period, the transfection mixture was replaced with serum-containing growth medium and maintained under normal growth conditions until the cells were assayed for firefly luciferase expression 24 hours after the initial treatment.

For fluorescence imaging, a similar transfection protocol was performed on U87-GFP cells seeded onto an 8-well LabTek coverglass chamber (Nalge Nunc, Naperville, Ill.) at a density of 5000 cells/well. U87-GFP cells were treated with a Cy5-labeled siRNA to facilitate imaging of cellular localization of siRNA.

### Fluorescence Microscopy:

Uptake of a fluorescently labeled siRNA (Dharmacon, Lafayette, Colo.) sequence into U87-GFP cells was evaluated using fluorescence microscopy. Imaging was performed 24 hours after siRNA transfection using an Olympus IX81 model fluorescent microscope (Olympus, Center Valley, Pa.). Imaging was performed at 20× magnification. The following excitation and emission wavelengths were used: GFP (excitation=482 nm, emission=536 nm) and Cy5 siRNA (excitation=628 nm, emission=692 nm).

### Luciferase Detection Assay:

Cells were prepared for firefly luciferase detection using the Luciferase Assay System (Promega, Madison, Wis.) according to the manufacturer's protocol. Firefly luciferase was quantified using The Reporter microplate luminometer (Turner Biosystems, Sunnyvale, Calif.). Following luciferase quantification, cell lysates were assayed for total protein content using the BCA Protein Assay kit (Pierce, Rockford, Ill.) according to the manufacturer's protocol.

### Statistics:

Statistical comparisons for zeta potential measurements, luciferase silencing and polymer cytotoxicity were performed using a one-way ANOVA test with a Fisher's all-pairs post hoc comparison test.

### Results and Discussion:

AMs (e.g. polymers) were modified with two different lengths of ethyleneimine chains to yield three polymer systems: ethylenediamine to yield 1N, or pentaethylenehexamine to yield 5N and 9N polymers. The polymers were synthesized as shown in Scheme B from the amine-specific N-hydroxysuccinimide (NHS)-activated polymer 1, which has been the focus of a previous publication (J. Djordjevic, L. Del Rosario, J. Wang, K. Uhrich, Journal of Bioactive and Compatible Polymers 2008, 23, 532; E. Chnari, J. Nikitczuk, J. Wang, K. Uhrich, P. Moghe, Biomacromolecules 2006, 7, 1796.)

The parent compound, 3, served as the basic building block for the polymer modifications. Specifically, the carboxylic acid on the mucic acid backbone was activated with N-hydroxysuccinimide to functionalize the polymers with linear ethyleneimines, systematically increasing the total number of amines in the final polymers from one, 1N, up to nine, 9N. For 1N and 5N, an excess of the diamines coupled with their slow addition to the polymer solution via syringe pump were utilized to control for the disubstitution of polymer to both primary amines. For 9N, a 2:1 molar ratio of compound 4 to pentaethylene hexamine coupled with the slow addition of 4 to the diamine via syringe pump were used to limit the formation of undesired oligomers. Subsequently, a 1:1 molar ratio of the NHS-PEG with respect to 4 coupled with its slow addition to the diaminated 4 via syringe pump were utilized to limit the coupling of PEG to both sides of the 4. For all aminated polymers, isolation of cationic AMs with amines conjugated to, rather than associated with, the polymer was insured by precipitation from diethyl ether; this process precipitates the AM products but not the ethyleneimine starting materials. Amine conjugation was further verified by ¹H NMR spectroscopy. In addition to monitoring ¹H NMR spectra for the disappearance of protons associated with the NHS activating group (˜2.8 ppm), new peaks assigned to the ethyleneamine protons were observed resonating at 1.3 and 1.8 ppm for 1N and from 2.5-3.0 ppm for 5N and 9N. For all cationic AMs, the integrations of the ¹H NMR are consistent with mono PEG substitution to produce the desired, cationic AMs. The molecular weights of the cationic AMs were determined by GPC relative to PEG standards. As shown in Table 1 below, 1N and 5N have similar molecular weights while the molecular weight of 9N is approximately 1 kDa less. This difference can be attributed to the use of different PEG starting materials from different vendors with varying peak molecular weights. In addition, due to the incorporation of amines between the hydrophobic and hydrophilic component in the structure of 9N, the polymer may associate more with the column than the other cationic polymers, thereby making the molecular weight appear lower than it actually is. For all cationic AMs, the absence of a high molecular weight peak in the GPC corresponding to ˜10 kDa suggests there was little-to-no PEG di-substitution in any of the resulting polymers.

TABLE 1
Molecular weights, poydispersity indices (PDI), and
melting temperature (Tₘ) ethyleneimine-modified AMs

| Cationic AM | M_{W} (kDa) | PDI | Tₘ (° C.) |
|---|---|---|---|
| 1N | 6.3 | 1.1 | 58 |
| 5N | 6.4 | 1.1 | 59 |
| 9N | 5.5 | 1.1 | 59 |

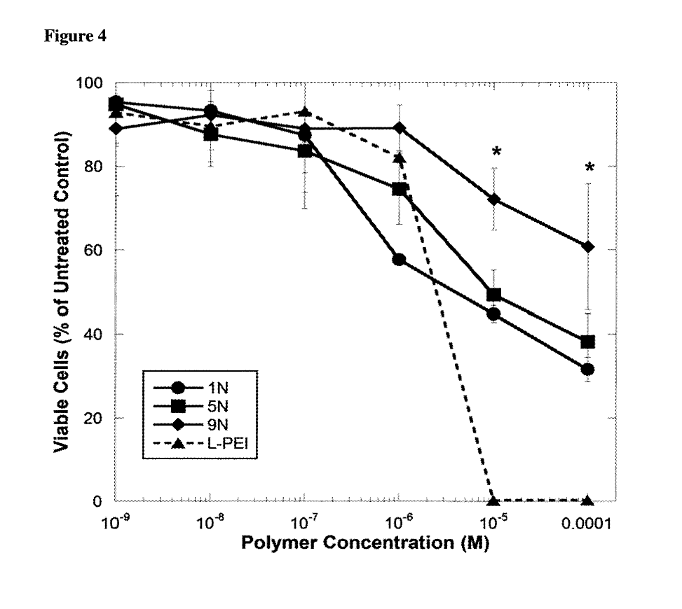

The toxicity of the aminated-AMs compared to linear PEI 25 kDa (L-PEI) was assessed in U87 glioma cells. A dose response curve was generated for all samples by counting viable cells remaining after a 72-hour exposure to the polymers (FIG. 4). A significant decrease in cytotoxicity (p<0.05) was observed for all AMs compared to L-PEI at the highest concentrations tested (10⁻⁵ and 10⁻⁴ M). Interestingly, when comparing the AMs, the 9N material exhibited the lowest cytotoxicity compared to the AMs containing fewer amine groups. One possible explanation for this observation is that the surface charge density of the 9N micelles is lower as their hydrodynamic diameter (shown in FIG. 5c) has increased by a factor of approximately five and, therefore, their area is increased by a factor of about 25 compared to the 1N and 5N micelles.

The ability of cationic AMs to complex anionic siRNA was evaluated using gel electrophoresis. Complexes were formed at a range of N/P ratios and run on an electrophoresis gel to separate un-complexed siRNA from the AM/siRNA complexes. The decrease in fluorescence intensity of the band corresponding to un-complexed siRNA verifies the siRNA complexation efficiency of the cationic AMs. Polymers containing zero or one cationic amine group (i.e., 1 and 1N) displayed no complexation of siRNA at charge ratios tested up to nitrogen/phosphorus (N/P) 80 (FIG. 5b). By increasing the number of amine groups to five (5N), a modest extent of siRNA complexation (approximately 20%) was observed at N/P ratios of 60 and higher. Significantly improved siRNA complexation efficiency was observed by using the AM containing nine amine groups, 9N, where most of the siRNA was encapsulated by N/P=50. The ability of AMs to complex siRNA was compared to L-PEI, where nearly complete siRNA complexation was observed by gel electrophoresis at N/P≧15. Hence, for subsequent physical and biological characterization studies, AM/siRNA complexes were formed at N/P≧50.

All cationic AMs formed micelles in the nanoscale size range as determined by dynamic light scattering (FIG. 5c). 1N and 5N formed micelles of approximately the same size as polymer, 1, while micelles formed from 9N were much larger (˜125 nm), presumably due to charge repulsion of the highly cationic ethyleneimine units. Once complexed with siRNA (FIG. 5c, N/P=50 and 100), all cationic AMs maintained the nanoscale size of the AMs alone. Self-assembled polymeric micelles are known to have stable sizes that are dictated primarily by the architecture of the amphiphilic polymer segments (K. Kataoka, A. Harada, Y. Nagasaki, Advanced Drug Delivery Review 2001, 47, 113.) Especially at high N/P ratios, the size of polymer micelles often remains unchanged in the presence of nucleic acids as the presence of relatively small amounts of siRNA does not change the properties of the stable polymer micelles (K. Kataoka, A. Harada, Y. Nagasaki, Advanced Drug Delivery Review 2001, 47, 113; S. Matsumoto, R. Christie, N. Nishiyama, K. Miyata, A. Ishii, M. Oba, H. Koyama, Y. Yamasaki, K. Kataoka, Biomacromolecules 2009, 10, 119). Maintaining sizes of less than ˜100 nm is desirable for improved circulation time, passive tumor targeting by the enhanced permeation and retention (EPR) effect, and optimal cellular uptake (L. Brannon-Peppas, J. O. Blanchette, Advance Drug Delivery Reviews 2004, 56, 1649; M. E. Davis, Z. Chen, D. M. Shin, Nature Reviews Druge Discovery 2008, 7, 771).

Successful conjugation of the amines was shown by the disappearance of N-hydroxysuccinimide in the ¹H NMR as well as the increase in the zeta potential from negative (for polymer 1), to less negative (polymer 1N), and positive (polymers 5N and 9N), as shown in FIG. 5d (micelles only). The zeta potential increased with increasing ethyleneimine length, further indicating the successful incorporation of amine groups. When siRNA was complexed with the cationic AMs at N/P ratios of 50 and 100, the zeta potentials for the aminated polymers 5N and 9N significantly changed compared to the native polymer in the absence of siRNA (p<0.05). Specifically, at N/P 50, the zeta potentials for complexes of siRNA and 5N decreased from 12.7 mV of the 5N alone to 5.3 mV when complexed with siRNA. Likewise, the zeta potential of 9N decreased from 33.1 mV alone to 7.89 mV when complexed with siRNA. The zeta potentials for both AMs increased at N/P 100—back to that for the vehicle alone for 5N but only to 22.2 mV for 9N. This data suggests that 9N complexed most efficiently with siRNA at the N/P ratios evaluated in this study, as the decrease in zeta potential is a result of charge neutralization when the negatively charged siRNA complexes with the cationic AMs. These results are in agreement with the gel electrophoresis data. Based on the physical characterization of AM/siRNA complexes by gel electrophoresis, dynamic light scattering, and zeta potential, 9N was expected to be the most effective siRNA delivery vehicle.

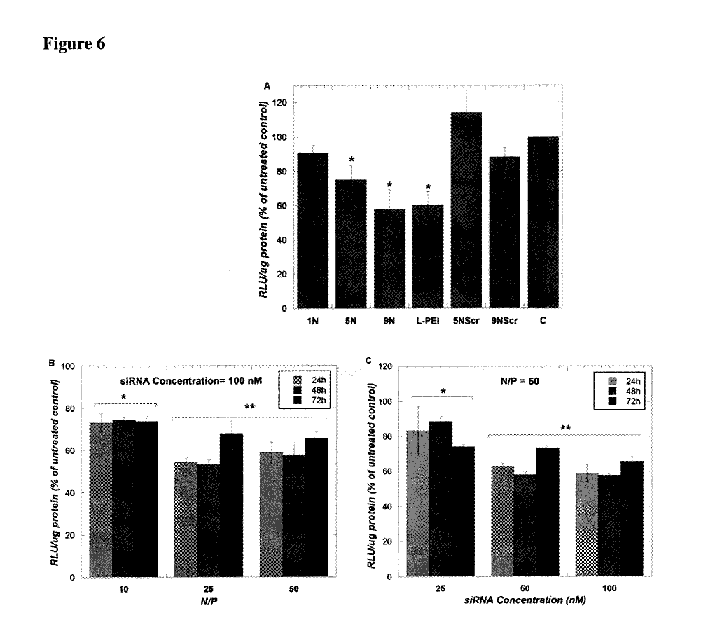

The ability of AMs to facilitate cellular delivery of siRNA and elicit silencing of the reporter gene, firefly luciferase, in U87 cells was evaluated. Polyplexes of AMs and anti-luciferase siRNA were formed (siRNA concentration: 100 nM, N/P=50, AM concentration: ˜10⁻⁵ M) and delivered to U87-Luc cells which were subsequently assayed for luciferase expression. To visually evaluate the cellular uptake of siRNA, a fluorescently labeled siRNA sequence was delivered separately to U87-GFP cells which were then imaged using fluorescent microscopy Significant luciferase silencing (p<0.05) was observed using the AMs containing five or nine amines (5N and 9N), but not observed using the AMs containing just one amine group (1N) (FIG. 6A). A similar luciferase silencing response was observed between the 9N and L-PEI, a widely-studied polymeric system for nucleic acid delivery (FIG. 6A). Delivering a scrambled siRNA sequence did not elicit luciferase silencing, demonstrating that the AMs alone do not induce off-target silencing effects.

To study the dynamics and dose-dependence of luciferase silencing by 9N, siRNA transfection experiments were performed at various time points, polymer concentrations (FIG. 6B) and siRNA concentrations (FIG. 6C). The minimum N/P ratio required for a maximum luciferase silencing response was N/P=25 (siRNA concentration: 100 nM, AM concentration: 1.2×10⁻⁵ M), (FIG. 6B), and the minimum siRNA concentration required for optimal silencing response was 50 nM (FIG. 6C).

Studying the dose response and dynamics of siRNA delivery by AMs provides insights into the mechanisms governing siRNA delivery by these novel molecules. The results suggest that using polymer 9N at N/P>25 may not be as biologically beneficial as similar extents of gene silencing were observed using 9N at N/P=25 and N/P=50. One goal in polymeric delivery systems is to identify the lowest possible polymer concentration that can achieve optimal siRNA delivery, as having excessively high polymer concentrations can elicit undesirable cytotoxicity and may result in insolubility of polymers in aqueous media. Further, it was observed that using siRNA concentrations of 50 nM was sufficient to achieve maximal luciferase gene silencing with 9N. Possibly, having siRNA in excess of the minimum effective concentrations is unnecessary as the number of target mRNAs present in the cell is limited; the cells are sufficiently targeted by siRNA at 50 nM.

Trends in siRNA silencing as a function of time, where maximum siRNA silencing was observed at 48 hours, and decreased after 72 hours were also observed. This trend in gene silencing dynamics is consistent with previous work evaluating the gene silencing dynamics of antisense oligonucleotides delivered by branched PEIs where the silencing of green fluorescent protein (GFP) became less pronounced after 24 hours (S. Sundaram, L. K. Lee, C. M. Roth, Nucleic Acids Res 2007, 35, 439690). This decrease in gene silencing activity after 48 hours may be attributed to intracellular degradation of siRNA molecules by nucleases over time.

The trends observed in the quantitative luciferase silencing assay differed somewhat from the qualitative observations of cellular association of a fluorescently labeled siRNA sequence into U87GFP cells. It appeared that 9N delivered more siRNA to the cells than L-PEI in the fluorescent images, however, this trend was not observed in the luciferase silencing assay where both 9N and L-PEI elicited similar extents of luciferase silencing. This observation may suggest that while 9N may be capable of delivering siRNA to cells, other intracellular barriers such as siRNA unpackaging or endosomal escape may be affecting gene silencing activity by 9N.

By increasing the number of secondary amines from one up to nine (i.e., from 1N to 9N, respectively), increased zeta potential and stable complexation with siRNA was achieved. All cationic AMs were less cytotoxic to U87 cells than L-PEI at polymer concentrations of 10 μM or greater.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

What is claimed is:

1. A compound of formula I: 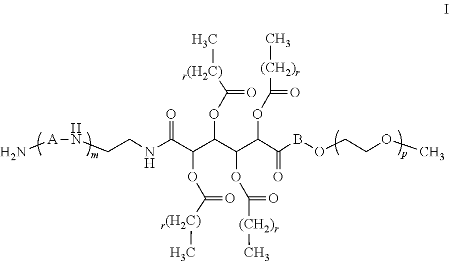
wherein:
B is absent or the group —NH(-A-NH—)ₙ—(CH₂)₂—NH—C(═O)—CH₂—;
each A is independently an ethylene group that is optionally substituted with one or more (C₁-C₃)alkyl groups;
m is an integer from 0 to 100;
n is an integer from 0 to 100;
p is an integer from 1 to 200; and
each r is independently an integer from 0 to 20;
or a salt thereof.

2. The compound of claim 1 wherein the compound of formula I is a compound formula I′: 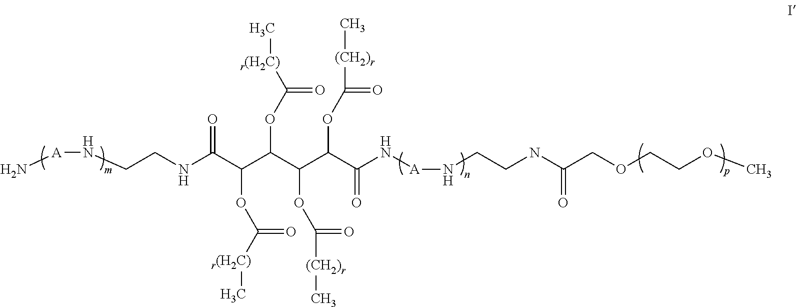
wherein:
each A is independently an ethylene group that is optionally substituted with one or more (C₁-C₃)alkyl groups;
m is an integer from 0 to 100;
n is an integer from 0 to 100;
p is an integer from 1 to 200; and
each r is independently an integer from 0 to 20;
or a salt thereof.

3. The compound of claim 1 wherein the compound of formula I is a compound formula I′: 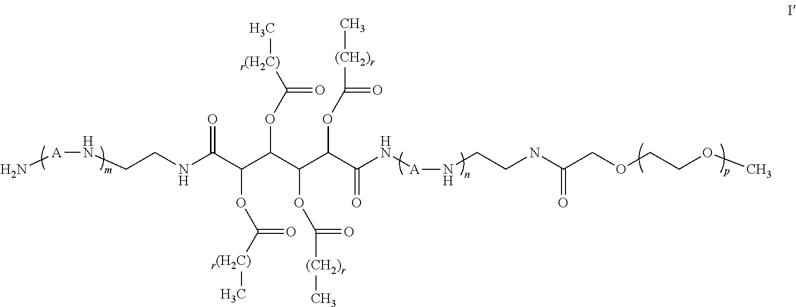
wherein: each A is independently an ethylene group that is optionally substituted with one or more (C₁-C₃)alkyl groups;
m is an integer from 1 to 100;
n is an integer from 1 to 100;
p is an integer from 20 to 200; and
each r is independently an integer from 0 to 20;
or a salt thereof.

4. The compound of claim 1 wherein the compound of formula I is a compound of formula Ia: 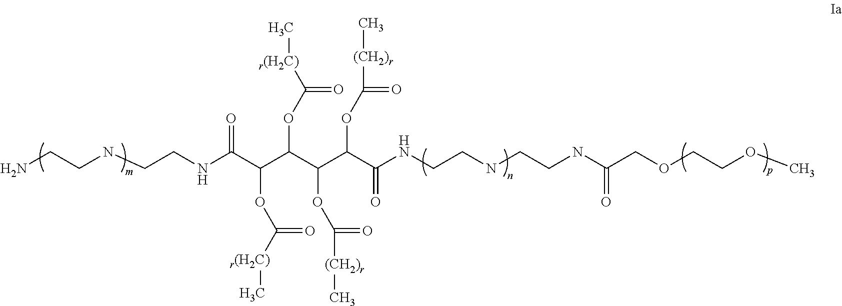
wherein:
m is an integer from 0 to 100;
n is an integer from 0 to 100;
p is an integer from 1 to 200; and
each r is independently an integer from 0 to 20;
or a salt thereof.

5. The compound of claim 1 wherein the compound of formula I is a compound of formula Ia: 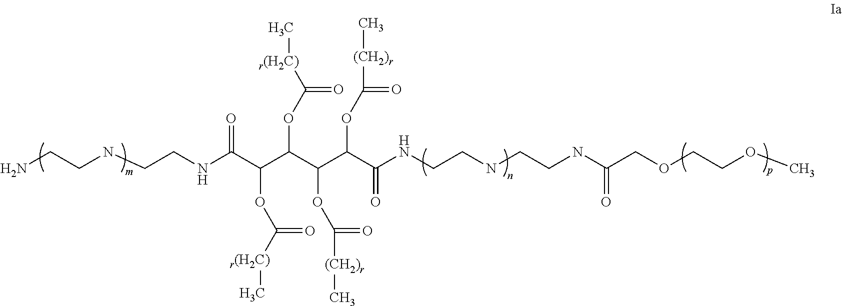
wherein:
m is an integer from 1 to 100;
n is an integer from 1 to 100;
p is an integer from 20 to 200; and
each r is independently an integer from 0 to 20;
or a salt thereof.

6. The compound of claim 1 wherein m is an integer from 1 to 20 and n is an integer from 1 to 20.

7. The compound of claim 1 wherein m is 1, 2, 3, or 4.

8. The compound of claim 1 wherein n is 1, 2, 3, or 4.

9. The compound of claim 1 wherein p is an integer from 20 to 200.

10. The compound of claim 1 wherein p is about 109.

11. The compound of claim 1 wherein each r is independently an integer from 0 to 20.

12. The compound of claim 1 wherein each r is independently 6, 7, 8, 9, 10, 11, 12, 13, 14, or 15.

13. The compound of claim 1 wherein: m is 2, 3, 4, or 5; n is 2, 3, 4, or 5; p is an integer from about 95 to about 125; and each r is independently 6, 7, 8, 9, 10, 11, 12, 13, 14, or 15.

14. The compound of claim 1 which is not a compound of the following formula: 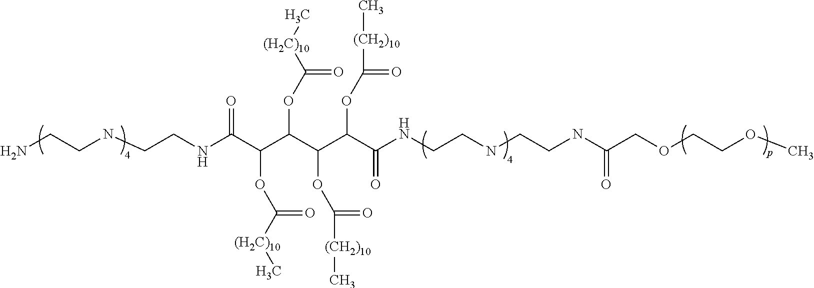
or a salt thereof.

15. The compound of claim 1 wherein the compound of formula I is a compound of formula Ib: 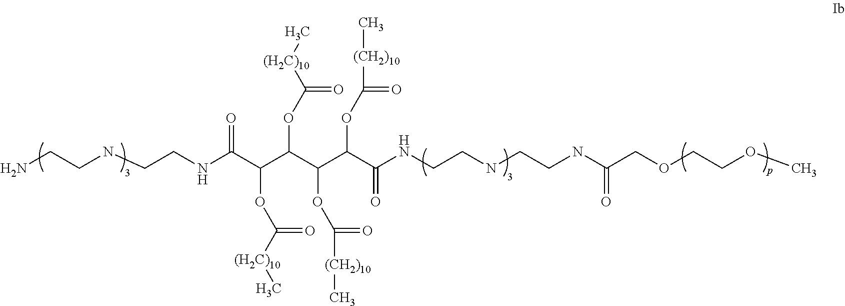
or a salt thereof.

16. A compound of claim 1 which is: 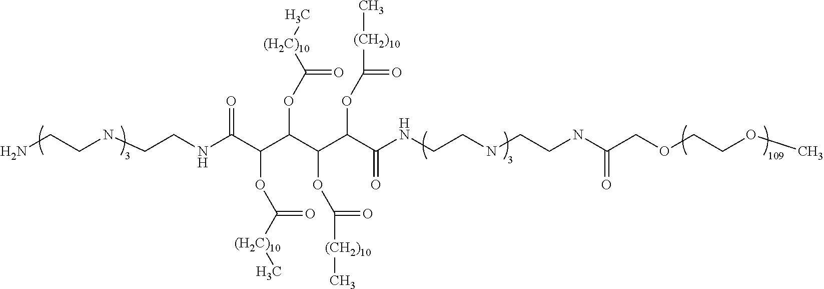
or a salt thereof.

17. A composition comprising a compound of formula I or a salt thereof as described in claim 1 and a nucleic acid.

18. The composition of claim 17 further comprising one or more lipids.

19. A method for delivering a nucleic acid into a cell comprising contacting the cell with a composition comprising a compound of formula I or a salt thereof as described in claim 1 and the nucleic acid under conditions such that the nucleic acid is delivered into the cell.

20. The method of claim 19 wherein the composition further comprises one or more lipids.
